(19) United States
(12) Patent Application Publication
(10) Pub. No.: US 2023/0176077 A1
(43) Pub. Date: Jun. 8, 2023

(54) RET (REARRANGED DURING TRANSFECTION) FOR THE ASSESSMENT OF STROKE

(71) Applicants: Universiteit Maastricht, Maastricht (NL); Academisch Ziekenhuis Maastricht, Maastricht (NL); Roche Diagnostics Operations, Inc., Indianapolis, IN (US)

(72) Inventors: Peter Kastner, Penzberg (DE); Vinzent Rolny, Penzberg (DE); Ursula-Henrike Wienhues-Thelen, Penzberg (DE); André Ziegler, Rotkreuz (CH); Manuel Dietrich, Mannheim (DE); Ulrich Schotten, Maastricht (NL)

(21) Appl. No.: 17/912,776

(22) PCT Filed: Mar. 18, 2021
(86) PCT No.: PCT/EP2021/056970

§ 371 Date: Sep. 19, 2022

(30) Foreign Application Priority Data
Mar. 18, 2020 (EP) 20164013.3

(51) Int. Cl. G01N 33/68

(52) U.S. Cl. CPC G01N 33/6896; G01N 2800/2871; G01N 2800/326; G01N 2800/50; G01N 2333/9121

(57) ABSTRACT

The present invention relates to a method for aiding in the prediction of stroke and/or dementia in a subject, said method comprising a) determining the amount of the biomarker RET (Rearranged during transfection) in a sample from the subject, b) comparing the amount determined in step a) to a reference, and c) aiding in the prediction of stroke and/or dementia. The present invention further relates to a method for aiding in the assessment of the extent of white matter lesions in a subject, a method for aiding in the assessment whether a subject has experienced one or more silent strokes and to a method for aiding in the diagnosis of atrial fibrillation in a subject. Further encompassed by the present invention are the corresponding uses.

The present invention relates to a method for aiding in the prediction of stroke and/or dementia in a subject, said method comprising a) determining the amount of the biomarker RET (Rearranged during transfection) in a sample from the subject, b) comparing the amount determined in step a) to a reference, and c) aiding in the prediction of stroke and/or dementia. The present invention further relates to a method for aiding in the assessment of the extent of white matter lesions in a subject, a method for aiding in the assessment whether a subject has experienced one or more silent strokes and to a method for aiding in the diagnosis of atrial fibrillation in a subject. Further encompassed by the present invention are the corresponding uses.

### BACKGROUND SECTION

Stroke ranks after ischemic heart disease second as a cause of lost disability-adjusted—life years in high income countries and as a cause of death worldwide. In order to reduce the risk of stroke, anticoagulation therapy appears the most appropriate therapy.

Atrial fibrillation (AF) is an important risk factor for stroke (Hart et al., Ann Intern Med 2007; 146(12): 857-67, Go A S et al. JAMA 2001; 285(18): 2370-5). Atrial fibrillation is characterized by irregular heart beating and often starts with brief periods of abnormal beating that can increase over time and may become a permanent condition. An estimated 2.7-6.1 million people in the United States have atrial fibrillation and approximately 33 million people globally (Chugh S. S. et al., Circulation 2014; 129-837-47). An early diagnosis of atrial fibrillation and an early prediction of the risk of stroke is highly desired because atrial fibrillation is an important risk factor for stroke and systemic embolism (Hart et al., Ann Intern Med 2007; 146(12): 857-67; Go A S et al. JAMA 2001; 285(18): 2370-5).

The diagnosis of heart arrhythmia such as atrial fibrillation typically involves determination of the cause of the arrhythmia, and classification of the arrhythmia. Guidelines for the classification of atrial fibrillation according to the American College of Cardiology (ACC), the American Heart Association (AHA), and the European Society of Cardiology (ESC) are mainly based on simplicity and clinical relevance. The first category is called “first detected AF”. People in this category are initially diagnosed with AF and may or may not have had previous undetected episodes. If a first detected episode stops on its own in less than one week, but is followed by another episode later on, the category changes to “paroxysmal AF”. Although patients in this category have episodes lasting up to 7 days, in most cases of paroxysmal AF the episodes will stop in less than 24 hours. If the episode lasts for more than one week, it is classified as “persistent AF”. If such an episode cannot be stopped, i.e. by electrical or pharmacologic cardioversion, and continues for more than one year, the classification is changed to “permanent AF”.

Recent evidence suggests that patients with AF also face an increased risk of cognitive dysfunction and dementia (Conen et al., J Am Coll Cardiol 2019; 73:989-99). Part of the association between AF and dementia is explained by the higher stroke risk among patients with AF, but the risk of dementia was also increased in patients with AF but without a clinical history of stroke Clinically unrecognized cerebral infarcts (i.e. silent cerebral infarcts) or other brain lesions, such as white matter lesions might explain the association. White matter refers to areas of the central nervous system (CNS) that are predominantly made up of myelinated axons. The extent of white matter lesions can be expressed by the Fazekas score (Fazekas, J B Chawluk, A Alavi, H I Hurtig, and R A Zimmerman American Journal of Roentgenology 1987 149:2, 351-356). The Fazekas score is ranging from 0 to 3.0 indicates no WML, 1 mild WML, 2 moderate WML and 3 severe WML.

White matter lesions (WML) on magnetic resonance imaging are linked to several adverse outcomes, such as cognitive impairment and depression. For example, white matter changes have been reported to be associated with a decline in motor function in speed and fine motor coordination, and with many diseases including vascular dementia, dementia with Lewy bodies, and psychiatric disorders. Further, it has been shown that the severity of dementia is significantly associated with white matter changes in Alzheimer's disease patients (Kao et al., J. Clin. Med. 2019, 8, 167; doi:10.3390/jcm8020167). In contrast, global microstructural integrity of the normal appearing white matter did not differ between Parkinson's disease patients and healthy control subjects (de Schipper et al., Neurobiology of Aging 80 (2019) 203-209).

Biomarkers which allow for the prediction of the risk of stroke and/or dementia and for the diagnosis of atrial fibrillation are highly required.

Rearranged during transfection (RET) is a proto-oncogene tyrosine-protein kinase receptor (UniProtKB—P07949).

RET has been proposed to be involved in many cellular mechanisms both during and post-development, including cell proliferation, cell migration, cell differentiation, haemapotoesis and neuronal navigation.

RET is a neurotrophic factor receptor, that drives both neuron function and also haematopoietic stem cell (HSC) survival and function (Mulligan, Nature Rev. Cancer 2014, Vol 14, pp 173, Fonseca-Pereira et al., Nature, 2014, Vol 514, pp 98). HSCs are quiescent in adulthood but can become proliferative upon physiological demand Autonomic nerves have been shown to be in close proximity to HSCs.

Säleby et al. measured 28 biomarkers in venous plasma from patients with pulmonary arterial hypertension (PAH). One of the measured biomarkers was RET. It was shown that the biomarker was decreased in subjects with PAH (Säleby et al, ERJ Open Res 2019; 5: 00037-2019).

The role of RET as an oncogenic driver in several cancers has been well described as a critical determinant of invasion and spread in diverse tumor types (Mulligan, Nature Rev. Cancer 2014, Vol. 14, pp 173). Due to the importance of RET in diverse cancers, it has been also described as a valuable therapeutic target (Mulligan, Frontiers in Physiology, 2019, Vol. 9, pp 1873).

In animal models, RET ablation was observed to impair gut homeostasis and to increase the risks of inflammation or infection in the gut (lbiza et al., Nature, 2016, Vol. 535, pp 440-443).

In further animal models GDNF and RET were described to be up-regulated in the brain in response to ischemia (Arvidsson et al., Neuroscience 2001, Vol 106, pp 27; Sarabi et al., Neurosci Lett 2003, Vol. 341, pp 241).

Rydberg et al. describe a cytokine profiling in the prefrontal cortex of Parkinson's Disease and Multiple System Atrophy patients. RET mRNA levels were increased in brain tissue from patients with both Parkinson's Disease (PD) and Multiple System Atrophy patients compared with normal controls (Rydberg et al., Neurobiology of Disease 106 (2017) 269-278) Further, it was shown that RET expression persists in human substantia nigra neurons in Parkinson's disease (Walker et al., Brain Research 792 1998, 207-217).

However, so far reduced circulating RET levels have not yet been described for the assessment of the risk of stroke, or for the assessment of the extent of white matter lesions or for the assessment of atrial fibrillation.

Advantageously, it has been found in the studies underlying the present invention that RET is a biomarker for the prediction of the stroke and/or dementia. Moreover, the level of RET is decreased in patients with atrial fibrillation. Therefore, the present invention allows for predicting the risk of stroke and/or dementia, for example, in patients with atrial fibrillation based on the amount of RET in a sample, such as blood, serum or plasma sample. The determination of RET further allows for improving the prediction of clinical accuracy of clinical stroke risk scores.

Further, it was shown that the biomarker RET negatively correlates with existence of white matter lesions (WML) in patients. Since WML extent can be caused by clinically silent strokes (Wang Y, Liu G, Hong D, Chen F, Ji X, Cao G. White matter injury in ischemic stroke. Prog Neurobiol. 2016; 141:45-60), the biomarker RET can be used for the assessment of the extent of white matter lesions and for the assessment whether a subject has experienced one or more silent strokes, i.e. clinically silent strokes, in the past.

### BRIEF SUMMARY OF THE PRESENT INVENTION

The present invention relates to a method for aiding in the prediction of stroke and/or dementia in a subject, said method comprising
a) determining the amount of the biomarker RET (Rearranged during transfection) in a sample from the subject,
b) comparing the amount determined in step a) to a reference, and
c) aiding in the prediction of stroke and/or dementia.

The present invention further relates to the in vitro use of the biomarker RET or of an agent which binds to the biomarker RET for aiding in the prediction of stroke and/or dementia in a subject.

In a preferred embodiment of the aforementioned methods and the aforementioned use, the subject suffers from atrial fibrillation.

In a preferred embodiment of the aforementioned methods and the aforementioned use, the risk of stroke is predicted, such as the risk of ischemic stroke.

In a preferred embodiment of the aforementioned methods and the aforementioned use, the risk of dementia is predicted such as the risk of vascular dementia, Alzheimer's disease, dementia with Lewy bodies, and/or frontotemporal dementia.

In a preferred embodiment of the aforementioned methods and the aforementioned use, an amount of RET lower than the reference is indicative for a subject who is at risk of stroke and/or dementia, and/or an amount of RET larger than the reference is indicative for a subject who is not at risk of stroke and/or dementia.

In a preferred embodiment of the aforementioned methods and the aforementioned use, the risk of the subject to suffer from stroke and/or dementia within 1 to 10 years is predicted.

The present invention further relates to a method for aiding in the assessment of the extent of white matter lesions in a subject, said method comprising,
a) determining the amount of the biomarker RET (Rearranged during transfection) in a sample from the subject, and
b) aiding in the assessment of the extent of white matter lesions in a subject based on the amount determined in step a).

The present invention further relates to the in vitro use of the biomarker RET or of an agent which binds to the biomarker RET for aiding in the assessment of the extent of white matter lesions in a subject.

In a preferred embodiment of the aforementioned method and the aforementioned use, the subject suffers from atrial fibrillation.

The present invention further relates to a method for aiding in the assessment whether a subject has experienced one or more silent strokes, said method comprising
a) determining the amount of the biomarker RET (Rearranged during transfection) in a sample from the subject,
b) comparing the amount determined in step a) to a reference, and
c) aiding in the assessment whether a subject has experienced one or more silent strokes.

The present invention further relates to the in vitro use of the biomarker RET or of an agent which binds to the biomarker RET for aiding in the assessment whether a subject has experienced one or more silent strokes.

In a preferred embodiment of the aforementioned method and the aforementioned use, the subject suffers from atrial fibrillation.

In a preferred embodiment of the aforementioned method and the aforementioned use, an amount of RET lower than the reference is indicative for a subject who has experienced one or more silent strokes, and/or an amount of RET larger than the reference is indicative for a subject who has not experienced silent strokes.

The present further concerns a method for monitoring a subject (e.g. a subject who suffers from atrial fibrillation), comprising
a) determining the amount of the biomarker RET (Rearranged during transfection) in a first sample from the subject,
b) determining the amount of the biomarker RET (Rearranged during transfection) in a second sample from the subject which has been obtained after the first sample,
c) comparing the amount of the biomarker RET in the first sample to the amount of the biomarker RET in the second sample, and
d) monitoring the subject based on the results of step c).

Also, the present invention relates to the in vitro use of the biomarker RET or of an agent which binds to the biomarker RET for monitoring a subject (e.g. a subject who suffers from atrial fibrillation).

In some embodiments, the extent of white matter lesions in the subject and/or the cognitive function of the subject is monitored. In some embodiments, the second sample has been obtained at least 6 months after the first sample. In some embodiments, a decreased amount of the biomarker RET in the second sample as compared to first sample is indicative for an increase of the extent of white matter lesions in the subject and/or for a decline of the cognitive function of the subject.

The present invention further relates to a method for aiding in the diagnosis of atrial fibrillation in a subject, said method comprising
a) determining the amount of the biomarker RET (Rearranged during transfection) in a sample from the subject,
b) comparing the amount determined in step a) to a reference, and
c) aiding in the diagnosis of atrial fibrillation.

The present invention further relates to the in vitro use of the biomarker RET or of an agent which binds to the biomarker RET for aiding in the diagnosis of atrial fibrillation in a subject.

In a preferred embodiment of the aforementioned methods and the aforementioned use, the agent fibrillation is paroxysmal or persistent atrial fibrillation.

In a preferred embodiment of the aforementioned methods and the aforementioned use, the subject was in sinus rhythm at the time the sample has been obtained.

The present invention further relates to a method for improving the prediction accuracy of a clinical stroke risk score for a subject, comprising the steps of
a) determining the amount of the biomarker RET (Rearranged during transfection) in a sample from the subject, and
b) combining a value for the amount of the biomarker RET with the clinical stroke risk score, whereby the prediction accuracy of said clinical stroke risk score is improved.

The present invention further relates to the in Vitro use of the biomarker RET or of an agent which binds to the biomarker RET for improving the prediction accuracy of a clinical stroke risk score for a subject.

In a preferred embodiment of the methods and the use of the present invention, the subject has no known history of stroke and/or TIA (transient ischemic attack).

In a preferred embodiment of the methods and the uses of the present invention, the subject is 65 years or older.

In a preferred embodiment of the methods and the uses of the present invention, the sample is a body fluid sample, such as a blood, serum or plasma sample.

In another preferred embodiment of the methods and the uses of the present invention, the sample is tissue sample.

In a preferred embodiment of the methods and the uses of the present invention, the biomarker RET is the RET polypeptide.

In a preferred embodiment of the methods and the uses of the present invention, the subject is a human subject.

### DETAILED DESCRIPTION OF THE PRESENT INVENTION—DEFINITIONS

As set forth above, the present invention relates to a method for aiding in the prediction of stroke and/or dementia in a subject, said method comprising
a) determining the amount of the biomarker RET (Rearranged during transfection) in a sample from the subject,
b) comparing the amount determined in step a) to a reference, and
c) aiding in the prediction of stroke and/or dementia.

In a preferred embodiment, the prediction in step b) is based on the amount of the biomarker in the sample, i.e. based on the comparison step b).

The methods as referred to in accordance with the present invention includes methods which essentially consist of the aforementioned steps or methods which include further steps. Moreover, the method of the present invention, preferably, is an ex vivo) and more preferably an in vitro method. Moreover, it may comprise steps in addition to those explicitly mentioned above. For example, further steps may relate to the determination of further markers and/or to sample pre-treatments or evaluation of the results obtained by the method. The method may be carried out manually or assisted by automation. Preferably, step (a), (b) and/or (c) may in total or in part be assisted by automation. e.g., by a suitable robotic and sensory equipment for the determination in step (a) or a computer-implemented calculation in step (b).

As will be understood by those skilled in the art, the assessments as described herein, such as the prediction of stroke and/or dementia, the assessment of the extent of white matter lesions, the assessment whether a subject has experienced one or more silent strokes, the diagnosis of atrial fibrillation in a subject, and the improvement of the prediction accuracy of a clinical stroke risk score for a subject are usually not intended to be correct for 100% of the subjects. In an embodiment, the prediction can be made for a statistically significant portion of subjects in a proper and correct manner. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research. John Wiley & Sons, New York 1983 Preferred confidence intervals are at least 90%, at least 95%, at least 97%, at least 98%, or at least 99%. The p-values are, preferably, 0.1, 0.05, 0.01, 0.005, or 0.0001.

The “subject” to be tested in accordance with the methods and use of the present invention, preferably, is a mammal Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the subject is a human subject. The terms “subject” and “patients” are used interchangeably herein.

In an embodiment of the methods and uses of the present invention, the subject is 65 years of age or older. In another embodiment, the subject is 70 years of age or older. In another embodiment, the subject is 75 years of age or older.

In a preferred embodiment of the method and uses of the present invention, the subject to be tested suffers from atrial fibrillation. Atrial fibrillation may be paroxysmal, persistent or permanent atrial fibrillation. Thus, the subject may suffer from paroxysmal, persistent or permanent atrial fibrillation. In particular, it is envisaged that the subject suffers from paroxysmal, persistent or permanent atrial fibrillation. The best performance was observed in patients with persistent atrial fibrillation.

Thus, in an embodiment of the present invention, the subject suffers from paroxysmal atrial fibrillation. In another embodiment of the present invention, the subject suffers from persistent atrial fibrillation. In another embodiment of the present invention, the subject suffers from permanent atrial fibrillation.

The term “Atrial Fibrillation” is well known in the art. As used herein, the term preferably refers to a supraventricular tachyarrhythmia characterized by uncoordinated atrial activation with consequent deterioration of atrial mechanical function. In particular, the term refers to an abnormal heart rhythm characterized by rapid and irregular beating. It involves the two upper chambers of the heart. In a normal heart rhythm, the impulse generated by the sino-atrial node spreads through the heart and causes contraction of the heart muscle and pumping of blood. In atrial fibrillation, the regular electrical impulses of the sino-atrial node are replaced by disorganized, rapid electrical impulses which result in irregular heart beats Symptoms of atrial fibrillation are heart palpitations, fainting, shortness of breath, or chest pain. However, most episodes have no symptoms. On the electrocardiogram atrial fibrillation is characterized by the replacement of consistent P waves by rapid oscillations or fibrillatory waves that vary in amplitude, shape, and timing, associated with an irregular, frequently rapid ventricular response when atrioventricular conduction is intact.

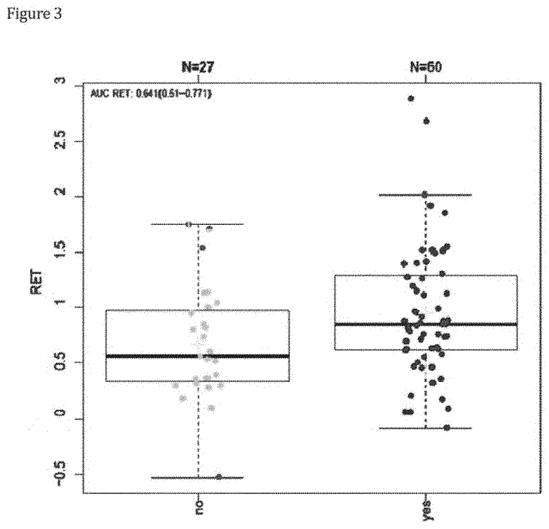

The American College of Cardiology (ACC), American Heart Association (AHA), and the European Society of Cardiology (ESC) propose the following classification system (see Fuster (2006) Circulation 114 (7): e257-354 which herewith is incorporated by reference in its entirety, see e.g. FIG. 3 in the document): First detected AF, paroxysmal AF, persistent AF, and permanent AF.

All people with AF are initially in the category called first detected AF. However, the subject may or may not have had previous undetected episodes. A subject suffers from permanent AF, if the AF has persisted for more than one year. In particular, conversion back to sinus rhythm does not occur (or only with medical intervention). A subject suffers from persistent AF, if the AF lasts more than 7 days. The subject may require either pharmacologic or electrical intervention to terminate atrial fibrillation. Thus persistent AF occurs in episodes, but the arrhythmia does not typically convert back to sinus rhythm spontaneously (i.e. without medical invention). Paroxysmal atrial fibrillation, preferably, refers to an intermittent episode of atrial fibrillation which lasts not longer than 7 days and terminates spontaneously (i.e. without medical intervention). In most cases of paroxysmal AF, the episodes last less than 24 hours. Thus, whereas paroxysmal atrial fibrillation terminates spontaneously, persistent atrial fibrillation does not end spontaneously and requires electrical or pharmacological cardioversion for termination, or other procedures, such as ablation procedures (Fuster (2006) Circulation 114 (7) e257-354). The term “paroxysmal atrial fibrillation” is defined as episodes of AF that terminate spontaneously in less than 48 hours, more preferably in less than 24 hours, and, most preferably in less than 12 hours. Both persistent and paroxysmal AF may be recurrent.

As set forth above, the subject to be tested preferably suffers from paroxysmal, persistent or permanent atrial fibrillation.

In an embodiment, the subject suffers from an episode of atrial fibrillation at the time when the sample is obtained. This may be e.g. the case if the subject suffers from permanent or persistent AF. In another embodiment, the subject does not suffer from an episode of atrial fibrillation at the time when the sample is obtained. This may be e.g. the case, if the subject suffers from persistent or paroxysmal AF. Accordingly, the subject may be a patient who suffers from atrial fibrillation, but who has a normal sinus rhythm when the sample is obtained, i.e. is in sinus rhythm. Thus, it is envisaged that the subject is in sinus rhythm at the time the sample has been obtained.

Further, it is contemplated that the atrial fibrillation has been diagnosed previously in the subject. Accordingly, the atrial fibrillation shall be a diagnosed, i.e. a detected, atrial fibrillation.

Further, it is envisaged that the subject to be tested in accordance with the methods and use of the present invention, may have no known history of stroke and/or TIA (transient ischemic attack).

In an embodiment, the subject has no known history of stroke. In another embodiment, the subject has no known history of both stroke and TIA. Thus, the subject to be tested shall not have suffered from clinically recognized strokes and/or TIAs.

The term “sample” refers to a sample of a body fluid, to a sample of separated cells or to a sample from a tissue or an organ. Samples of body fluids can be obtained by well-known techniques and include samples of blood, plasma, serum, urine, lymphatic fluid, sputum, ascites, saliva, lacrimal fluid, cerebrospinal fluid or any other bodily secretion or derivative thereof. Tissue or organ samples may be obtained from any tissue or organ by, e.g., biopsy. Separated cells may be obtained from the body fluids or the tissues or organs by separating techniques such as centrifugation or cell sorting. E.g., cell-, tissue- or organ samples may be obtained from those cells, tissues or organs which express or produce the biomarker. For example, the sample may be a myocardial tissue sample. Further, the sample may be a neural tissue sample, or a gut tissue sample. In some embodiments, the sample is a bone marrow sample. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample.

Thus, the sample may be a tissue sample. In a preferred embodiment, the tissue sample is a heart tissue sample, such as a myocardial tissue sample. In particular, the sample is a tissue sample from the right atrial appendage. In another preferred embodiment, the sample is a neural tissue sample, such as a brain tissue sample or spinal cord sample.

In another preferred embodiment, the sample is a blood (i.e. whole blood), serum or plasma sample. For example, the sample may be venous blood, serum or plasma sample. Alternatively, the sample may be a capillary blood sample (e.g. obtained from a finger). In some embodiments, the sample is peripheral blood sample. Serum is the liquid fraction of whole blood that is obtained after the blood is allowed to clot. For obtaining the serum, the clot is removed by centrifugation and the supernatant is collected. Plasma is the acellular fluid portion of blood. For obtaining a plasma sample, whole blood is collected in anticoagulant-treated tubes (e.g. citrate-treated or EDTA-treated tubes). Cells are removed from the sample by centrifugation and the supernatant (i.e. the plasma sample) is obtained.

Further, the sample may comprise stem cells, such as stem cells from the bone marrow or peripheral blood, lymphocytes, cardiomyocytes, neuronal cells or gut cells.

In some embodiments, the sample is a cerebrospinal fluid sample.

The term “predicting the risk” as used herein, preferably, refers to assessing the probability according to which the subject will suffer from stroke and/or dementia Typically, it is predicted whether a subject is at risk (and thus at elevated risk) or not at risk (and thus at reduced risk) of suffering from stroke and/or dementia. Accordingly, the method of the present invention allows for differentiating between a subject at risk and a subject not at risk of suffering from stroke and/or dementia. Further, it is envisaged that the method of the present invention allows for differentiating between a subject who is a reduced, average, or elevated risk. The term “aiding”, preferably means that further measures are taken into account for the assessment described herein, e.g. the determination of further biomarkers, or the confirmation of the assessment by further means, such as MRI.

As set forth above, the risk (and probability) of suffering from stroke and/or dementia within a certain time window shall be predicted. In an embodiment of the present invention, the predictive window, preferably, is an interval at least 1 month, at least 3 months, at least 6 months, at least 9 months, at least 1 year, at least 2 years, at least 3 years, at least 4 years, at least 5 years, at least 10 years, at least 15 years, or at least 20 years, or any inter-mitting time range Preferably, the predictive window is a period of about three years. Also preferably, the predictive window might be a period of about five years.

In a preferred embodiment, the predictive window a period of 1 to 3 years Thus, the risk to suffer from stroke and/or dementia within 1 to 3 year is predicted. In a preferred embodiment, the predictive window a period of 1 to 10 years. Thus, the risk of the subject to suffer from stroke and/or dementia within 1 to 10 years is predicted.

Preferably, said predictive window is calculated from the completion of the method of the present invention. More preferably, said predictive window is calculated from the time point at which the sample to be tested has been obtained.

In a preferred embodiment, the expression “predicting the risk of stroke and/or dementia” means that the subject to be analyzed by the method of the present invention is allocated either into the group of subjects being at risk of suffering from stroke and/or dementia, or into the group of subjects not being at risk of suffering from stroke and/or dementia. Thus, it is predicted whether the subject is at risk or not at risk of suffering from stroke and/or dementia. As used herein “a subject who is at risk of suffering from stroke and/or dementia”, preferably has an elevated risk of suffering from stroke and/or dementia (preferably within the predictive window). Preferably, said risk is elevated as compared to the average risk in a cohort of subjects. As used herein, “a subject who is not at risk of suffering from stroke and/or dementia”, preferably, has a reduced risk of suffering from stroke and/or dementia (preferably within the predictive window) Preferably, said risk is reduced as compared to the average risk in a cohort of subjects. A subject who is at risk of suffering from stroke and/or dementia preferably has a risk of suffering from stroke and/or dementia of at least 20% or more preferably of at least 30%, preferably, within a predictive window of about three years. A subject who is not at risk of suffering from stroke and/or dementia preferably has a risk of lower than 12%, more preferably of lower than 10% of suffering from said adverse event, preferably within a predictive window of three years.

In a preferred embodiment, the risk of stroke is predicted. The term “stroke” is well known in the art. The term, preferably, refers to ischemic stroke, in particular to cerebral ischemic stroke. A stroke which is predicted by the method of the present invention shall be caused by reduced blood flow to the brain or parts thereof which leads to an undersupply of oxygen to brain cells. In particular, the stroke leads to irreversible tissue damage due to brain cell death. Symptoms of stroke are well known in the art. Ischemic stroke may be caused by atherothrombosis or embolism of a major cerebral artery, by coagulation disorders or nonatheromatous vascular disease, or by cardiac ischemia which leads to a reduced overall blood flow. The ischemic stroke is preferably selected from the group consisting of atherothrombotic stroke, cardioembolic stroke and lacunar stroke. The term “stroke” does, preferably, not include hemorrhagic stroke.

Whether a subject suffers from stroke, in particular from ischemic stroke can be determined by well-known methods. Moreover, symptoms of stroke are well known in the art. E.g., stroke symptoms include sudden numbness or weakness of face, arm or leg, especially on one side of the body, sudden confusion, trouble speaking or understanding, sudden trouble seeing in one or both eyes, and sudden trouble walking, dizziness, loss of balance or coordination.

In a preferred embodiment of the aforementioned methods and the aforementioned use, the risk of dementia is predicted. Alternatively, it may be predicted whether a subject is at risk of cognitive decline, or not.

The term “dementia” as used herein, preferably, refers to a condition which can be characterized as a loss, usually progressive, of cognitive and intellectual functions, without impairment of perception or consciousness caused by a variety of disorders, but most commonly associated with structural brain disease. The most common type of dementia is Alzheimer's disease, which makes up 50% to 70% of cases Other common types include vascular dementia (25%), dementia with Lewy bodies, and frontotemporal dementia. The term 2. “dementia” includes, but is not restricted to AIDS dementia, Alzheimer dementia, presenile dementia, senile dementia, catatonic dementia, Lewy body dementia (diffuse Lewy body disease), multi-infarct dementia (vascular dementia), paralytic dementia, posttraumatic dementia, dementia praecox, vascular dementia.

In an embodiment, the term dementia refers to vascular dementia, Alzheimer's disease, dementia with Lewy bodies, and/or frontotemporal dementia Thus, the risk to suffer from vascular dementia, Alzheimer's disease, dementia with Lewy bodies, and/or frontotemporal dementia is predicted.

In an embodiment, the risk to suffer from “Alzheimer's disease” is predicted. The term “Alzheimer's disease” is well known in the art. Alzheimer's disease is a chronic neuro-degenerative disease that usually starts slowly and gradually worsens over time. As the disease advances, symptoms can include problems with language, disorientation, mood swings, loss of motivation, not managing self-care, and behavioural issues.

In an embodiment, the risk to suffer from “vascular dementia” is predicted. The term “vascular dementia” preferably refers to progressive loss of memory and other cognitive functions caused by vascular injury or disease within the brain. Thus, the term shall refer to the symptoms of dementia caused by problems of circulation of blood to the brain. It may occur after a stroke or build up over time.

The methods of the present invention can be also used for the screening of larger populations of subjects. Therefore, it is envisaged, that at least 100 subjects, in particular at least 1000 subjects are assessed, e.g. with respect to the risk of stroke. Thus, the amount of the biomarker RET is determined in samples from at least 100, or in particular of from at least 1000 subjects. Moreover, it is envisaged that at least 10,000 subjects are assessed.

The biomarker “Rearranged during transfection” (abbreviated RET) is well-known in the art. Rearranged during transfection (RET) is a proto-oncogene tyrosine-protein kinase receptor (UniProtKB—P07949). The RET polypeptide, i.e. the proto-oncogene tyrosine-protein kinase receptor Ret, is involved in numerous cellular mechanisms including cell proliferation, neuronal navigation, cell migration, and cell differentiation upon binding with glial cell derived neurotrophic factor family ligands. Further, it regulates both cell death/survival balance and positional information. Alternative names for RET are “Cadherin family member 12” and “Proto-oncogene c-Ret”. Preferably, the biomarker RET is human RET. The sequence of the RET transcripts and the RET polypeptide are well-known in the art and can be assessed via UniProt under the accession number UniProtKB—P07949, via Entrez (Gene ID: 5979, updated on 3 Feb. 2020), or Ensembl (ENSG00000165731).

In a preferred embodiment of the methods and the uses of the present invention, the biomarker RET is the RET polypeptide. Thus, the amount of the RET polypeptide is determined.

In a preferred embodiment of the methods and the uses of the present invention, the biomarker RET is RET mRNA Thus, the amount RET mRNA is determined (which can be done directly or indirectly).

The term “determining” the amount of a biomarker as referred to herein (such as RET) refers to the quantification of the biomarker, e.g. to measuring the level of the biomarker in the sample, employing appropriate methods of detection described elsewhere herein. The terms “measuring” and “determining” are used herein interchangeably.

In an embodiment, the amount of a biomarker is determined by contacting the sample with an agent that specifically binds to the biomarker, thereby forming a complex between the agent and said biomarker, detecting the amount of complex formed, and thereby measuring the amount of said biomarker.

The biomarkers as referred to herein can be detected using methods generally known in the art. Methods of detection generally encompass methods to quantify the amount of a biomarker in the sample (quantitative method). It is generally known to the skilled artisan which of the following methods are suitable for qualitative and/or for quantitative detection of a biomarker. Samples can be conveniently assayed for, e.g., proteins using Westerns and immunoassays, like ELISAs, RIAs, fluorescence- and luminescence-based immunoassays and proximity extension assays, which are commercially available. Further suitable methods to detect biomarkers include measuring a physical or chemical property specific for the peptide or polypeptide such as its precise molecular mass or NMR spectrum. Said methods comprise, e.g., biosensors, optical devices coupled to immunoassays, biochips, analytical devices such as mass-spectrometers, NMR-analyzers, or chromatography devices. Further, methods include microplate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

For the detection of biomarker proteins as referred to herein a wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker.

Methods employing electrochemiluminescent labels are well-known. Such methods make use of the ability of special metal complexes to achieve, by means of oxidation, an excited state from which they decay to ground state, emitting electrochemiluminescence. For review see Richter, M. M., Chem. Rev. 2004; 104: 3003-3036.

In an embodiment, the detection antibody (or an antigen-binding fragment thereof) to be used for measuring the amount of a biomarker is ruthenylated or iridinylated. Accordingly, the antibody (or an antigen-binding fragment thereof) shall comprise a ruthenium label. In an embodiment, said ruthenium label is a bipyridine-ruthenium(II) complex. Or the antibody (or an antigen-binding fragment thereof) shall comprise an iridium label. In an embodiment, said iridium label is a complex as disclosed in WO 2012/107419.

In an embodiment of the sandwich assay for the determination of RET, the assay comprises a biotinylated first monoclonal antibody that specifically binds RET (as capture antibody) and a ruthenylated F(ab′)2-fragment of a second monoclonal antibody that specifically binds RET as detection antibody) The two antibodies form sandwich immunoassay complexes with RET in the sample.

Measuring the amount of a polypeptide may, preferably, comprise the steps of (a) contacting the polypeptide with an agent that specifically binds said polypeptide, (b) (optionally) removing non-bound agent, (c) measuring the amount of bound binding agent, i.e. the complex of the agent formed in step (a). According to a preferred embodiment, said steps of contacting, removing and measuring may be performed by an analyzer unit. According to some embodiments, said steps may be performed by a single analyzer unit of said system or by more than one analyzer unit in operable communication with each other. For example, according to a specific embodiment, said system disclosed herein may include a first analyzer unit for performing said steps of contacting and removing and a second analyzer unit, operably connected to said first analyzer unit by a transport unit (for example, a robotic arm), which performs said step of measuring.

The agent which specifically binds the biomarker (herein also referred to as “binding agent”) may be coupled covalently or non-covalently to a label allowing detection and measurement of the bound agent. Labeling may be done by direct or indirect methods. Direct labeling involves coupling of the label directly (covalently or non-covalently) to the binding agent. Indirect labeling involves binding (covalently or non-covalently) of a secondary binding agent to the first binding agent. The secondary binding agent should specifically bind to the first binding agent. Said secondary binding agent may be coupled with a suitable label and/or be the target (receptor) of a tertiary binding agent binding to the secondary binding agent. Suitable secondary and higher order binding agents may include antibodies, secondary antibodies, and the well-known streptavidin-biotin system (Vector Laboratories, Inc.). The binding agent or substrate may also be “tagged” with one or more tags as known in the art. Such tags may then be targets for higher order binding agents.

Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium complexes, iridium complexes, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase. Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, available as ready-made stock solution from Roche Diagnostics), CDP-Star™ (Amersham Biosciences), ECF™ (Amersham Biosciences). A suitable enzyme-substrate combination may result in a colored reaction product, fluorescence or chemoluminescence, which can be determined according to methods known in the art (e.g. using a light-sensitive film or a suit-able camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. A radioactive label can be detected by any method known and appropriate, e.g. a light-sensitive film or a phosphor imager.

The amount of a polypeptide may be, also preferably, determined as follows: (a) contacting a solid support comprising a binding agent for the polypeptide as described elsewhere herein with a sample comprising the peptide or polypeptide and (b) measuring the amount of peptide or polypeptide which is bound to the support. Materials for manufacturing supports are well-known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc.

In yet another aspect the sample is removed from the complex formed between the binding agent and the at least one marker prior to the measurement of the amount of formed complex. Accordingly, in an aspect, the binding agent may be immobilized on a solid support. In yet another aspect, the sample can be removed from the formed complex on the solid support by applying a washing solution.

“Sandwich assays” are among the most useful and commonly used assays encompassing a number of variations of the sandwich assay technique. Briefly, in a typical assay, an unlabeled (capture) binding agent is immobilized or can be immobilized on a solid substrate, and the sample to be tested is brought into contact with the capture binding agent. After a suitable period of incubation, for a period of time sufficient to allow formation of a binding agent-biomarker complex, a second (detection) binding agent labeled with a reporter molecule capable of producing a detectable signal is then added and incubated, allowing time sufficient for the formation of another complex of binding agent-biomarker-labeled binding agent. Any unreacted material may be washed away, and the presence of the biomarker is determined by observation of a signal produced by the reporter molecule bound to the detection binding agent. The results may either be qualitative, by simple observation of a visible signal, or may be quantitated by comparison with a control sample containing known amounts of biomarker.

The incubation steps of a typical sandwich assays can be varied as required and appropriate. Such variations include for example simultaneous incubations, in which two or more of binding agent and biomarker are co-incubated. For example, both, the sample to be analyzed and a labeled binding agent are added simultaneously to an immobilized capture binding agent. It is also possible to first incubate the sample to be analyzed and a labeled binding agent and to thereafter add an antibody bound to a solid phase or capable of binding to a solid phase.

The formed complex between a specific binding agent and the biomarker shall be proportional to the amount of the biomarker present in the sample. It will be understood that the specificity and/or sensitivity of the binding agent to be applied defines the degree of proportion of at least one marker comprised in the sample which is capable of being specifically bound. Further details on how the measurement can be carried out are also found elsewhere herein. The amount of formed complex shall be transformed into an amount of the biomarker reflecting the amount indeed present in the sample.

The terms “binding agent”, “specific binding agent”, “analyte-specific binding agent”, “detection agent”, “agent that binds to a biomarker” and “agent that specifically binds to a biomarker”, are used interchangeably herein. Preferably it relates to an agent that comprises a binding moiety which specifically binds the corresponding biomarker. Examples of “binding agents”, “detection agents”, “agents” are a nucleic acid probe, nucleic acid primer, DNA molecule, RNA molecule, aptamer, antibody, antibody fragment, peptide, peptide nucleic acid (PNA) or chemical compound. A preferred agent is an antibody which specifically binds to the biomarker to be determined. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity (i.e. antigen-binding fragments thereof). Preferably, the antibody is a polyclonal antibody (or an antigen-binding fragment thereof). More preferably, the antibody is a monoclonal antibody (or an antigen-binding fragment thereof). Moreover, as described elsewhere herein, it is envisaged that two monoclonal antibodies are used that bind at different positions of the RET polypeptide (in a sandwich immunoassay). Thus, at least one antibody is used for the determination of the amount of RET.

The agent or detection agent shall specifically bind the biomarker RET. The term “specific binding” or “specifically bind” refers to a binding reaction wherein binding pair molecules exhibit a binding to each other under conditions where they do not significantly bind to other molecules. The term “specific binding” or “specifically binds”, when referring to a protein or peptide as biomarker, preferably refers to a binding reaction wherein a binding agent binds to the corresponding biomarker with an affinity (“association constant” Kₐ) of at least 10⁷ M⁻¹. The term “specific binding” or “specifically binds” preferably refers to an affinity of at least 10⁸ M⁻¹ or even more preferred of at least 10⁹ M⁻¹ for its target molecule. The term “specific” or “specifically” is used to indicate that other molecules present in the sample do not significantly bind to the binding agent specific for the target molecule.

The term “amount” as used herein encompasses the absolute amount of a biomarker as referred to herein (such as RET), the relative amount or concentration of the said bi-marker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts determined from biological read out systems in response to the peptides or intensity signals obtained from specifically bound ligands. It is to be understood that values correlating to the aforementioned amounts or parameters can also be obtained by all standard mathematical operations.

The term “comparing” as used herein refers to comparing the amount of the biomarker (RET) in the sample from the subject with the reference amount of the biomarker specified elsewhere in this description. It is to be understood that comparing as used herein usually refers to a comparison of corresponding parameters or values, e.g., an absolute amount is compared to an absolute reference amount while a concentration is compared to a reference concentration, or an intensity signal obtained from the biomarker in a sample is compared to the same type of intensity signal obtained from a reference sample. The comparison may be carried out manually or computer-assisted. Thus, the comparison may be carried out by a computing device. The value of the determined or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provides the desired prediction in a suitable output format.

In accordance with the present invention, the amount of the biomarker RET shall be compared to a reference, i.e. to a reference amount (or to reference amounts). Accordingly, the reference is preferably a reference amount. The terms “reference amount” or “reference” are well understood by the skilled person. It is to be understood that the reference amount shall allow for aiding in the prediction of stroke and/or dementia, in the assessment of the extent of white matter lesions, in the assessment whether a subject has experienced one or more silent strokes, in the diagnosis of atrial fibrillation in a subject, and the improvement of the prediction accuracy of a clinical stroke risk score for a subject as described herein elsewhere. For example, in connection with the method for aiding in the prediction of the risk of stroke and/or dementia, the reference amount preferably refers to an amount which allows for allocation of a subject into either (i) the group of subjects who are at risk of suffering from stroke and/or dementia, or (ii) the group of subjects who are at risk of suffering from stroke and/or dementia. For example, in connection with the method for aiding in the diagnosis of atrial fibrillation, the reference amount preferably refers to an amount which allows for allocation of a subject into either (i) the group of subjects suffering from atrial fibrillation or (ii) the group of subjects not suffering from atrial fibrillation. A suitable reference amount may be determined from a reference sample to be analyzed together, i.e. simultaneously or subsequently, with the test sample.

Reference amounts can, in principle, be calculated for a cohort of subjects as specified above based on the average or mean values for a given biomarker by applying standard methods of statistics. In particular, accuracy of a test such as a method aiming to diagnose an event, or not, is best described by its receiver-operating characteristics (ROC) (see especially Zweig M H. et al., Clin. Chem. 1993; 39:561-577). The ROC graph is a plot of all the sensitivity versus specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus 1—specificity for the complete range of thresholds suitable for making a distinction. On the y-axis is sensitivity, or the true-positive fraction, which is defined as the ratio of number of true-positive test results to the product of number of true-positive and number of false-negative test results. It is calculated solely from the affected subgroup. On the x-axis is the false-positive fraction, or 1—specificity, which is defined as the ratio of number of false-positive results to the product of number of true-negative and number of false-positive results. It is an index of specificity and is calculated entirely from the unaffected subgroup Because the true- and false-positive fractions are calculated entirely separately, by using the test results from two different subgroups, the ROC plot is independent of the prevalence of the event in the cohort. Each point on the ROC plot represents a sensitivity/1—specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 450 diagonal line from the lower left corner to the upper right corner. Most plots fall in between these two extremes. If the ROC plot falls completely below the 45′ diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis for a given event with a proper balance of sensitivity and specificity, respectively. Accordingly, the reference to be used for the method of the present invention, i.e. a threshold which allows the respective assessment, such as the prediction of stroke and/or dementia, the assessment of the extent of white matter lesions, the assessment whether a subject has experienced one or more silent strokes, the diagnosis of atrial fibrillation in a subject, and the improvement of the prediction accuracy of a clinical stroke risk score for a subject, can be generated, preferably, by establishing a ROC for said cohort as described above and deriving a threshold amount therefrom Dependent on a desired sensitivity and specificity for the assessment, the ROC plot allows deriving a suitable threshold. It will be understood that an optimal sensitivity is desired for e.g. excluding a subject being at risk of stroke and/or dementia (i.e. a rule out) whereas an optimal specificity is envisaged for a subject to be predicted to be at risk of stroke and/or stroke (i.e. a rule in).

Since it is known in the art that the amount of RET decreases with age, an age-specific, i.e. age-matched, reference amount may be used. This is taken into account by the skilled person.

Preferably, the term “reference amount” herein refers to a predetermined value Said predetermined value shall allow for the assessment as referred to herein, such as the prediction of stroke and/or dementia, the assessment of the extent of white matter lesions, the assessment whether a subject has experienced one or more silent strokes, the diagnosis of atrial fibrillation in a subject, and the improvement of the prediction accuracy of a clinical stroke risk score for a subject.

In the method for aiding in the prediction of the risk of stroke and/or dementia, for example, the reference, i.e. the reference amount shall allow for differentiating between a subject who is at risk of suffering from stroke and/or dementia and a subject who is not at risk of suffering from stroke and/or dementia. In the method for aiding in the diagnosis of AF, for example, the reference shall allow for differentiating between a subject suffering from AF and a subject who is not suffering AF.

In connection with the prediction of the risk of stroke and/or dementia, the diagnostic algorithm is preferably as follows:

Preferably, an amount of RET lower than the reference is indicative for a subject who is at risk of stroke and/or dementia, and/or an amount of RET larger than the reference is indicative for a subject who is not at risk of stroke and/or dementia.

In the studies underlying the present invention, it has been further shown that the determination of RET allows for improving the prediction accuracy of a clinical stroke risk score for a subject. Thus, the combined determination of clinical stroke risk score and the amount of RET allows for an even more reliable prediction of stroke as compared to the determination of RET alone or the determination of the clinical stroke risk score alone. Moreover, risk scores recommended by ESC Guidelines are not sensitive enough and miss patients for anti-coagulation therapy.

Accordingly, the method for predicting the risk of stroke may further comprise the combination of the amount of RET with the clinical stroke risk score. Based on the combination of the amount of RET clinical risk score, the risk of stroke of the test subject is predicted.

Alternatively, the method may comprise obtaining or providing the value for the clinical stroke risk score. Preferably, the value is a number. In an embodiment, the clinical stroke risk score is generated by one of the clinically based tools available to physicians. Preferably, the value provided by determining the value for the clinical stroke risk score for the subject More preferably, the value for the subject is obtained from patient record databases and medical history of the subject. The value for the score therefore can be also determined using historical or published data of the subject.

In accordance with the present invention, the amount of RET is may be combined with the clinical stroke risk score. This means preferably that a value for the amount of RET is combined with the clinical stroke risk score. Accordingly, the values are operatively combined to predict the risk of the subject to suffer from stroke. By combining the value, a single value may be calculated, which itself can be used for the prediction.

Clinical stroke risk scores are well known in the art. E.g. said scores are described in Kirchhof P. et al., (European Heart Journal 2016; 37: 2893-2962). In an embodiment, the score is CHA₂DS₂-VASc-Score. In another embodiment, the score is the CHADS₂ Score. (Gage B F. Et al., JAMA, 285 (22) (2001), pp. 2864-2870) and ABC score, i.e. the ABC (age, biomarkers, clinical history) stroke risk score (Hijazi Z. et al., Lancet 2016; 387(10035): 2302-2311). All publications in this paragraph are herewith incorporated by reference with respect to their entire disclosure content.

Thus, in an embodiment, the clinical stroke risk score is the CHA₂DS₂-VASc-Score. In an alternative embodiment of the present invention, the clinical stroke risk score is the CHADS₂ Score. In a further embodiment, the clinical risk score is the ABC Score. The ABC stroke risk score is a novel biomarker-based risk score for predicting stroke in AF was validated in a large cohort of patients with AF and further externally validated in an independent AF cohort (see Hijazi et al., 2016, see above). It includes the age of the subject, the blood, serum or plasma levels of cardiac Troponin T and NT-proBNP in said subject, and information on whether the subject has a history of stroke Preferably, the ABC stroke score is the score as disclosed in Hijazi et al., 2016).

In a preferred embodiment, the above method for predicting the risk of stroke in a subject further comprises the step of recommending anticoagulation therapy or of recommending an intensification of anticoagulation therapy if the subject has been identified to be at risk to suffer from stroke (as described elsewhere herein).

The present invention further relates to a method for improving the prediction accuracy of a clinical stroke risk score for a subject, comprising the steps of
a) determining the amount of the biomarker RET (Rearranged during transfection) in a sample from the subject, and
b) combining a value for the amount of the biomarker RET with the clinical stroke risk score, whereby the prediction accuracy of said clinical stroke risk score is improved.

The method may comprise the further step of c) improving prediction accuracy of said clinical stroke risk score based on the results of step b).

The definitions and explanations given herein above in connection with the method of aiding in the prediction of the risk of stroke and/or dementia preferably apply to the aforementioned method as well. E.g., it envisaged that the subject is a subject who has a known clinical stroke risk score. Alternatively, the method may comprise obtaining or providing the value for the clinical stroke risk score.

In accordance with the aforementioned, the amount of RET is combined with the clinical stroke risk score. This means preferably, that the value for the amount of RET is combined with the clinical stroke risk score. Accordingly, the values are operatively combined to improve the prediction accuracy of said clinical stroke risk score.

The method of the present invention may aid personalized medicine. In a preferred embodiment, the method for predicting the risk of stroke in a subject further comprises i) the step of recommending anticoagulation therapy, or ii) of recommending an intensification of anticoagulation therapy, if the subject has been identified to be at risk to suffer from stroke. In another preferred embodiment, the method for predicting the risk of stroke in a subject further comprises i) the step of initiating anticoagulation therapy, or ii) of intensifying anti-coagulation therapy, if the subject has been identified to be at risk to suffer from stroke (by the method of the present invention).

Anticoagulation therapy is preferably a therapy which aims to reduce the risk of anticoagulation in said subject Administration of at least one anticoagulant shall aim to reduce or prevent coagulation of blood and related stroke. In a preferred embodiment, at least one anticoagulant is selected from the group consisting of heparin, a coumarin derivative (i.e. a vitamin K antagonist), in particular warfarin or dicumarol, oral anticoagulants, in particular dabigatran, rivaroxaban or apixaban, tissue factor pathway inhibitor (TFPI), antithrombin III, factor IXa inhibitors, factor Xa inhibitors, inhibitors of factors Va and VIIIa and thrombin inhibitors (anti-IIa type).

In a particularly preferred embodiment, the anticoagulant is a vitamin K antagonist such as warfarin or dicumarol. Vitamin K antagonists, such as warfarin or dicumarol are less expensive, but need better patient compliance, because of the inconvenient, cumbersome and often unreliable treatment with fluctuating time in therapeutic range. NOAC (new oral anticoagulants) comprise direct factor Xa inhibitors (apixaban, rivaroxaban, darexaban, edoxaban), direct thrombin inhibitors (dabigatran) and PAR-1 antagonists (vorapaxar, atopaxar).

If the test subject is on anticoagulation therapy, and if the subject has been identified not to be at risk to suffer from stroke (by the method of the present invention) the dosage of anticoagulation therapy may be reduced. Accordingly, a reduction of the dosage may be recommended. Be reducing the dosage, the risk to suffer from side effects (such as bleeding) may be reduced.

The term “recommending” as used herein means establishing a proposal for a therapy which could be applied to the subject. However, it is to be understood that applying the actual therapy whatsoever is not comprised by the term. The therapy to be recommended depends on the outcome of, e.g. of the prediction by the method of the present invention.

In particular, the following applies:

If the subject to be tested does not receive anticoagulation therapy, the initiation of anticoagulation is recommended, if the subject has been identified to be at risk to suffer from stroke. Thus, anticoagulation therapy shall be initiated.

If the subject to be tested already receives anticoagulation therapy, the intensification of anticoagulation is recommended, if the subject has been identified to be at risk to suffer from stroke. Thus, anticoagulation therapy shall be intensified.

In a preferred embodiment, anticoagulation therapy is intensified by increasing the dosage of the anticoagulant, i.e. the dosage of the currently administered coagulant.

In a particularly preferred embodiment, anticoagulation therapy is intensified by replacing the currently administered anticoagulant with a more effective anticoagulant. Thus, a replacement of the anticoagulant is recommended.

It has been described that better prevention in high risk patients is achieved with the oral anticoagulant apixaban versus the vitamin K antagonist warfarin as shown in Hijazi at al., The Lancet 2016 387, 2302-2311, (FIG. 4).

Thus, it is envisaged that the subject to be tested is a subject who is treated with a vitamin K antagonist such as warfarin or dicumarol. If the subject has been identified to be at risk to suffer from stroke (by the method of the present invention), the replacement of the vitamin K antagonist with an oral anticoagulant, in particular dabigatran, rivaroxaban or apixaban is recommended. Accordingly, the therapy with the vitamin K antagonist is discontinued and therapy with an oral anticoagulant is initiated.

The definitions given herein above, preferably, apply mutatis mutandis to the following method for aiding in the assessment of the extent of white matter lesions.

Interestingly, it was shown in the studies underlying the present invention that the biomarker RET can be used for estimating the risk, presence and/or severity of cerebrovascular injury as cause of dementia and cognitive dysfunction in atrial fibrillation patients. Specifically, it was shown that RET negatively correlates with existence of white matter lesions (WML) in patients. The lower the amount of RET, the higher the extent of white matter lesions (and vice versa) Therefore, RET can be used as a marker for assessing the extent of white matter lesions.

Accordingly, the present invention further relates to a method for aiding in the assessment of the extent of white matter lesions in a subject, said method comprising,
a) determining the amount of the biomarker RET (Rearranged during transfection) in a sample from the subject, and
b) aiding in the assessment of the extent of white matter lesions in a subject, preferably, based on the amount determined in step a).

The terms “subject” and “sample” have been defined above. The definitions apply accordingly. For example, it is envisaged that the subject suffers from atrial fibrillation. Further, the sample may be, for example, a blood, serum or plasma sample, or tissue sample.

The term “white matter lesions” is well-known in the art. White matter refers to areas of the central nervous system (CNS) that are predominantly made up of myelinated axons. White matter lesions (also referred to as “White matter disease”) is commonly detected on brain MRI of aging individuals as white matter hyperintensities (WMH), or ‘leukoaraiosis”. It has been described that the presence and extent of WMH is a radiographic marker of small cerebral vessel disease and an important predictor of the life-long risk of stroke, cognitive impairment, and functional disability (Chutinet A, Rost N S. White matter disease as a biomarker for long-term cerebrovascular disease and dementia. Curr Treat Options Cardiovasc Med. 2014; 16(3):292. doi:10.1007/s11936-013-0292-z). The determination of RET allows for assessing the extent of WMLs, i.e. the burden of WMLs. Accordingly, the biomarker allows for the quantification of WMLs in a subject, i.e. it is a marker for loss of volume of functional brain tissue.

The extent of white matter lesions can be expressed by the Fazekas score (Fazekas, J B Chawluk, A Alavi, H I Hurtig. and RA Zimmerman American Journal of Roentgenology 1987 149.2, 351-356). The Fazekas score is ranging from 0 to 3.0 indicates no WML, 1 mild WML, 2 moderate WML and 3 severe WML.

The definitions given herein above, preferably, apply mutatis mutandis to the following method.

WML extent can be caused by clinically silent strokes. Therefore, the biomarker RET could be further used for aiding in the assessment whether a subject has experienced one or more silent strokes in past, i.e. before the sample has been obtained.

Accordingly, the present invention further relates to a method for aiding in the assessment whether a subject has experienced one or more silent strokes, said method comprising
a) determining the amount of the biomarker RET (Rearranged during transfection) in a sample from the subject,
b) comparing the amount determined in step a) to a reference, and
c) aiding in the assessment whether a subject has experienced one or more silent strokes, preferably, based on the results of the comparison step.

Silent strokes, i.e. silent cerebral strokes, are known in the art and are, for example, described in Conen et al. (Conen et al., J Am Coil Cardiol 2019; 73:989-99) which herewith is incorporated by reference with respect to its entire disclosure content. Silent strokes are clinically silent strokes in patients without a clinical history of stroke or transient ischemic attack. Accordingly, the subject to be tested shall have no known history of stroke and/or TIA (transient ischemic attack).

In a preferred embodiment of the, the subject to be tested suffers from atrial fibrillation.

The following preferably applies as diagnostic algorithm:

An amount of RET lower than the reference is indicative for a subject who has experienced one or more silent strokes, and/or an amount of RET larger than the reference is indicative for a subject who has not experienced silent strokes.

The definitions given herein above, preferably, apply mutatis mutandis to the following: The studies carried out in the studies of the present invention indicate that it would be possible to monitor a subject based on changes in the amount of RET. For example, the extent of white matter lesions can be monitored, i.e. whether the volume of white matter lesions increases, or not. Since an increase of the extent of white matter lesions may be associated with a decrease of cognitive function, the determination of the biomarker RET would also allow for monitoring the cognitive function of a subject.

Accordingly, the present further concerns a method for monitoring a subject, comprising
a) determining the amount of the biomarker RET (Rearranged during transfection) in a first sample from the subject,
b) determining the amount of the biomarker RET (Rearranged during transfection) in a second sample from the subject which has been obtained after the first sample,
c) comparing the amount of the biomarker RET in the first sample to the amount of the biomarker RET in the second sample, and
d) monitoring the subject based on the results of step c).

Also present invention further relates to the in vitro use of the biomarker RET or of an agent which binds to the biomarker RET for monitoring a subject. In some embodiments, the biomarker RET or the agent is used in a first and a second sample from the subject.

The subject to be monitored may be a subject as defined in connection with the method for predicting the risk of stroke and/or dementia. For example, the subject may suffer from atrial fibrillation.

Preferably, the extent of white matter lesions in the subject and/or the cognitive function of the subject is monitored. However, it is also envisaged to monitor morphological changes of the myocardial atrium, cerebral infarctions, cerebral micro bleeds, progression of arrhythmia, progression of comorbidities (hypertension or diabetes) and/or progression of depressive symptoms. Alternatively, the amount of functional brain tissue may be monitored.

The monitoring shall be based on the comparison of the amount of the biomarker RET in a first sample to the amount of the biomarker RET in the second sample. The “second sample” is understood as a sample which is obtained in order to reflect a change of the amount of the biomarker RET as compared to the amount of RET in the first sample. Thus, second sample shall be obtained after the first sample. Preferably, the second sample is not obtained too early after the first sample (in order to observe a sufficiently significant change to allow for monitoring). In an embodiment, the second sample is obtained at least one month after the first sample. In another embodiment, the second sample is obtained at least six months after the first sample. In another embodiment, the second sample is obtained at least one or two years after the first sample. Further, it is envisaged that the second sample is obtained not more than 15 years, not more than 10 years, or, in particular, not more than five years after the first sample. Thus, the second sample may be obtained, e.g., at least six months, but not more than five years after the first sample.

Further, it is envisaged that the subject exhibited a stroke between the first and the second sample. The term “stroke” has been defined herein above.

Preferably, a decreased amount, in particular a significantly decreased amount of the biomarker RET in the second sample as compared to first sample is indicative for an increase of the extent of white matter lesions in the subject and/or for a decline of the cognitive function of the subject. Thus, the extent of white matter increased and/or the cognitive function declined between the first and the second sample. A significantly decreased amount of the biomarker RET is to be understood a decrease which is larger than the average decrease in a group of control subjects. In some embodiments, a decrease of the amount of the biomarker RET of at least 0.5% (e.g. per year) such as a decrease of at least 1% (e.g. per year), is indicative for an increase of the extent of white matter lesions and/or for a decline of the cognitive function.

The definitions given herein above, preferably, apply mutatis mutandis to the following:

Advantageously, it has been shown in the studies underlying the present invention that the determination of RET in a sample from a subject allows for the diagnosis of atrial fibrillation.

A method for aiding in the diagnosis of atrial fibrillation in a subject, said method comprising
a) determining the amount of the biomarker RET (Rearranged during transfection) in a sample from the subject,
b) comparing the amount determined in step a) to a reference, and
c) aiding in the diagnosis of atrial fibrillation.

The term “diagnosing” as used herein means assessing whether a subject as referred to in accordance with the present invention suffers from atrial fibrillation (AF), or not. In a preferred embodiment, it is diagnosed that a subject suffers from AF. In an alternative embodiment, it is diagnosed that a subject does not suffer from AF.

In accordance with the present invention, all types of AF can be diagnosed. Thus, the atrial fibrillation may be paroxysmal, persistent or permanent AF. Preferably, paroxysmal or persistent atrial fibrillation is diagnosed, for example in a subject not suffering from permanent AF. In some embodiments, persistent AF is diagnosed.

The actual diagnosis whether a subject suffers from AF, or not may comprise further steps such as the confirmation of a diagnosis (e.g. by ECG such as Holter-ECG). Thus, the present invention allows for assessing the likelihood that a patient suffers from atrial fibrillation. A subject who has an amount of RET below the reference amount is likely to suffer from atrial fibrillation, whereas a subject who has an amount of RET above the reference amount is not likely to suffer from atrial fibrillation. Accordingly, the term “diagnosing” in the context of the present invention also encompasses aiding the physician to assess whether a subject suffers from atrial fibrillation, or not.

In a preferred embodiment, the reference amount, i.e. the reference amount for RET shall allow for differentiating between a subject suffering from atrial fibrillation and a subject not suffering from atrial fibrillation. Preferably, said reference amount is a predetermined value.

Preferably, an amount of the RET biomarker in the sample from a test subject which is decreased as compared to the reference is indicative for a subject suffering from atrial fibrillation, and/or an amount of the RET biomarker in the sample from a subject which is increased as compared to the reference is indicative for a subject not suffering from atrial fibrillation.

The terms “subject” and “sample” have been defined above. The definitions apply accordingly. For example, it is envisaged that subject was in sinus rhythm at the time the sample has been obtained.

In an embodiment of the method of diagnosing atrial fibrillation, said method further comprises a step of recommending and/or initiating a therapy for atrial fibrillation based on the results of the diagnosis. Preferably, a therapy is recommended or initiated if it is diagnosed that the subject suffers from AF. Preferred therapies for atrial fibrillation are anticoagulation therapies as disclosed elsewhere herein.

A method for aiding in the diagnosis of the severity of dementia in a subject who suffers from dementia, said method comprising
a) determining the amount of the biomarker RET (Rearranged during transfection) in a sample from the subject,
b) comparing the amount determined in step a) to a reference, and
c) aiding in the diagnosis of the severity of dementia in a subject, preferably, based on the results of step c).

Diagnosis of cerebrovascular injury such as MWLs and/or clinically silent infarcts (including size, location and types of lesions) is nowadays performed using magnetic resonance imaging (MRI) that is typically lengthy and costly. The determination of RET, however would allow for a fast and cost-efficient pre-selection for cerebral MRI.

The methods of the present invention may further comprise the step of subjecting the patient who has been identified to be at risk of stroke or dementia, who has been identified to have a high extent of WMLs, who has been identified to have experienced one or more silent strokes in the past, and/or who has been diagnosed to suffer from AF, to Magnetic Resonance Imaging (MRI) of the brain, in particular to MRI for assessing cerebrovascular injury.

The present invention further relates to the in vitro use of the biomarker RET or of an agent which binds to the biomarker RET for aiding in the prediction of stroke and/or dementia in a subject.

The present invention further relates to the in vitro use of the biomarker RET or of an agent which binds to the biomarker RET for aiding in the assessment of the extent of white matter lesions in a subject.

The present invention further relates to the in vitro use of the biomarker RET or of an agent which binds to the biomarker RET for aiding in the assessment whether a subject has experienced one or more silent strokes.

The present invention further relates to the in vitro use of the biomarker RET or of an agent which binds to the biomarker RET for aiding in the diagnosis of atrial fibrillation in a subject.

The present invention further relates to the in vitro use of the biomarker RET or of an agent which binds to the biomarker RET for improving the prediction accuracy of a clinical stroke risk score for a subject.

Preferably, the aforementioned uses are in vitro uses. Thus, they are preferably carried out in a sample obtained from a subject. Moreover, the detection agent is, preferably, an antibody such as a monoclonal antibody (or an antigen binding fragment thereof) which specifically binds to the biomarker RET.

The methods of the present invention may be also carried out as computer-implemented methods.

Accordingly, the present invention relates to a computer-implemented method for aiding in the prediction of stroke and/or dementia in a subject, said method comprising
a) receiving at a processing unit a value for the amount of the biomarker RET (Rearranged during transfection) in a sample from the subject,
b) processing the value received in step (a) with the processing unit, wherein said processing comprises retrieving from a memory one or more threshold values for the amount of the biomarker RET and comparing the value received in step (a) with the one or more threshold values, and
c) providing a prediction of stroke and/or dementia via an output device, wherein said prediction is based on the results of step (b).

The present invention further relates to a computer-implemented method for aiding in the assessment of the extent of white matter lesions in a subject, said method comprising,
a) receiving at a processing unit a value for the amount of the biomarker RET (Rearranged during transfection) in a sample from the subject,
b) processing the value received in step (a) with the processing unit, wherein said processing comprises retrieving from a memory one or more threshold values for the amount of the biomarker RET and comparing the value received in step (a) with the one or more threshold values, and
c) providing an assessment of the extent of white matter lesions in a subject via an output device, wherein said assessment is based on the results of step (b).

The present invention further relates to a computer-implemented method for aiding in the assessment whether a subject has experienced one or more silent strokes, said method comprising
a) receiving at a processing unit a value for the amount of the biomarker RET (Rearranged during transfection) in a sample from the subject,
b) processing the value received in step (a) with the processing unit, wherein said processing comprises retrieving from a memory one or more threshold values for the amount of the biomarker RET and comparing the value received in step (a) with the one or more threshold values, and
c) providing an assessment whether a subject has experienced one or more silent strokes via an output device, wherein said assessment is based on the results of step (b).

The present invention further relates to a computer-implemented method for aiding in the diagnosis of atrial fibrillation in a subject, said method comprising
a) receiving at a processing unit a value for the amount of the biomarker RET (Rearranged during transfection) in a sample from the subject.
b) processing the value received in step (a) with the processing unit, wherein said processing comprises retrieving from a memory one or more threshold values for the amount of the biomarker RET and comparing the value received in step (a) with the one or more threshold values, and
c) providing a diagnosis of atrial fibrillation via an output device, wherein said diagnosis is based on the results of step (b).

In an embodiment of the methods of the present invention, information on the prediction, assessment, or diagnosis (according to the last step of the computer-implemented methods of the present invention) is provided via a display, configured for presenting the prediction, assessment, or diagnosis. For example, information may be provided whether the subject is at risk of stroke and/or dementia, or not. Further, recommendations for suitable therapeutic measures can be displayed.

In an embodiment of the methods of the present invention, the methods may comprise the further step of transferring the information on the assessment of the methods of the present invention to the subject's electronic medical records.

Alternatively, the assessment made in the last step of the methods of the present invention can be printed by a printer. The print-out shall contain information on whether the patient is at risk, or not at risk and/or a recommendation of a suitable therapeutic measure.

The present invention further relates to computer program including computer-executable instructions for performing the steps of the method according to the present invention, when the program is executed on a computer or computer network. Typically, the computer program specifically may contain computer-executable instructions for performing the steps of the method as disclosed herein. Specifically, the computer program may be stored on a computer-readable data carrier.

The present invention further relates to a computer program product with program code means stored on a machine-readable carrier, in order to perform the computer-implemented method according to present invention, such as the computer-implemented method for aiding in the prediction of stroke and/or dementia, when the program is executed on a computer or computer network, such as one or more of the above-mentioned steps discussed in the context of the computer program. As used herein, a computer program product refers to the program as a tradable product. The product may generally exist in an arbitrary format, such as in a paper format, or on a computer-readable data carrier. Specifically, the computer program product may be distributed over a data network.

The present invention further relates to a computer or computer network comprising at least one processing unit, wherein the processing unit is adapted to perform all steps of the computer-implemented method according to the present invention.

Yet, the present invention also contemplates:
A computer or computer network comprising at least one processing unit, wherein said processing unit is adapted to perform the method according to one of the embodiments described in this description,
a computer loadable data structure that is adapted to perform the method according to one of the embodiments described in this description while the data structure is being executed on a computer,
a computer script, wherein the computer program is adapted to perform the method according to one of the embodiments described in this description while the program being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network,
a computer program product having program code means, wherein the program code means can be stored or are stored on a storage medium, for performing the method according to one of the embodiments described in this description, if the program code means are executed on a computer or on a computer network,
a data stream signal, typically encrypted, comprising glucose data measurements obtained from the individual as specified herein above, and
a data stream signal, typically encrypted, comprising an information providing an aid in the assessment of guidance obtained by the method of the invention.

### EMBODIMENTS OF THE INVENTION

In the following, embodiments of the present invention are summarized. The definitions given herein above, preferably, apply to the following embodiments.
1. A method for aiding in the prediction of stroke and/or dementia in a subject, said method comprising
   a) determining the amount of the biomarker RET (Rearranged during transfection) in a sample from the subject,
   b) comparing the amount determined in step a) to a reference, and
   c) aiding in the prediction of stroke and/or dementia.
2. The method of embodiment 1, wherein the subject suffers from atrial fibrillation.
3. The method of embodiments 1 and 2, wherein the risk of stroke is predicted and wherein the stroke is ischemic stroke.
4. The method of embodiments 1 and 2, wherein the risk of dementia is predicted, and wherein dementia is vascular dementia, Alzheimer's disease, dementia with Lewy bodies and/or frontotemporal dementia
5. The method of any one of embodiments 1 to 4, wherein the biomarker RET is the RET polypeptide.
6. The method of any one of embodiments 1 to 5, wherein the sample is a blood, serum or plasma sample, or wherein the sample is tissue sample.
7. The method of any one of embodiments 1 to 6, wherein an amount of RET lower than the reference is indicative for a subject who is at risk of stroke and/or dementia, and/or wherein an amount of RET larger than the reference is indicative for a subject who is not at risk of stroke and/or dementia.
8. The method of any one of embodiments 1 to 7, wherein the subject has no known history of stroke and/or TIA (transient ischemic attack).
9. The method of any one of embodiments 1 to 8, wherein the risk of the subject to suffer from stroke and/or dementia in a subject within 1 to 10 years is predicted, such as within 3 years or within 5 years.
10. The method of any one of embodiments 1 to 9, wherein the subject is 65 years or older.
11. A method for aiding in the assessment of the extent of white matter lesions in a subject, said method comprising,
   a) determining the amount of the biomarker RET (Rearranged during transfection) in a sample from the subject,
   b) aiding in the assessment of the extent of white matter lesions in a subject based on the amount determined in step a).
12. The method of embodiment 11, wherein the subject suffers from atrial fibrillation.
13. The method of embodiments 11 and 12, wherein the sample is a blood, serum or plasma sample, or wherein the sample is tissue sample.
14. A method for aiding in the assessment whether a subject has experienced one or more silent strokes, said method comprising
   a) determining the amount of the biomarker RET (Rearranged during transfection) in a sample from the subject,
   b) comparing the amount determined in step a) to a reference, and
   c) aiding in the assessment whether a subject has experienced one or more silent strokes.
15. The method of embodiment 14, wherein the subject has no known history of stroke and/or TIA (transient ischemic attack)
16. The method of embodiments 14 and 15, wherein the subject suffers from atrial fibrillation.
17. The method of any one of embodiments 14 to 16, wherein the sample is a blood, serum or plasma sample, or wherein the sample is tissue sample
18. The method of any one of embodiments 14 to 17, wherein an amount of RET lower than the reference is indicative for a subject who has experienced one or more silent strokes, and/or wherein an amount of RET larger than the reference is indicative for a subject who has not experienced silent strokes.
19. A method for aiding in the diagnosis of atrial fibrillation in a subject, said method comprising
   a) determining the amount of the biomarker RET (Rearranged during transfection) in a sample from the subject,
   b) comparing the amount determined in step a) to a reference, and
   c) aiding in the diagnosis of atrial fibrillation.
20. The method of embodiment 19, wherein the atrial fibrillation is paroxysmal or persistent atrial fibrillation.
21. The method of embodiments 19 and 20, wherein the subject was in sinus rhythm at the time the sample has been obtained.
22. A method for improving the prediction accuracy of a clinical stroke risk score for a subject, comprising the steps of
   a) determining the amount of the biomarker RET (Rearranged during transfection) in a sample from the subject, and
   b) combining a value for the amount of the biomarker RET with the clinical stroke risk score, whereby the prediction accuracy of said clinical stroke risk score is improved.
23. In vitro use of the biomarker RET or of an agent which binds to the biomarker RET for aiding in the prediction of stroke and/or dementia in a subject.
24. In vitro use of the biomarker RET or of an agent which binds to the biomarker RET for aiding in the assessment of the extent of white matter lesions in a subject.
25. In vitro use of the biomarker RET or of an agent which binds to the biomarker RET for aiding in the assessment whether a subject has experienced one or more silent strokes.
26. In vitro use of the biomarker RET or of an agent which binds to the biomarker RET for aiding in the diagnosis of atrial fibrillation in a subject.
27. n vitro use of the biomarker RET or of an agent which binds to the biomarker RET for improving the prediction accuracy of a clinical stroke risk score for a subject.
28. A computer-implemented method for aiding in the prediction of stroke and/or dementia in a subject, said method comprising
   a) receiving at a processing unit a value for the amount of the biomarker RET (Rearranged during transfection) in a sample from the subject,
   b) processing the value received in step (a) with the processing unit, wherein said processing comprises retrieving from a memory one or more threshold values for the amount of the biomarker RET and comparing the value received in step (a) with the one or more threshold values, and
   c) providing a prediction of stroke and/or dementia via an output device, wherein said prediction is based on the results of step (b).

All references cited in this specification are herewith incorporated by reference with respect to their entire disclosure content and the disclosure content specifically mentioned in this specification.

In the Figures:

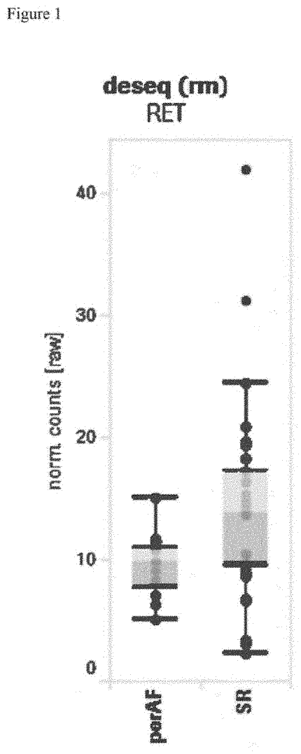

FIG. 1: Measurement of RET in Mapping study; Exploratory AFib panel; Patients with a history of atrial fibrillation undergoing open chest surgery and epicardial mapping of persistent AF (perAF) or Sinus Rhythm (SR), Mapping study). Atrial tissue RNA expression profiles was assessed.

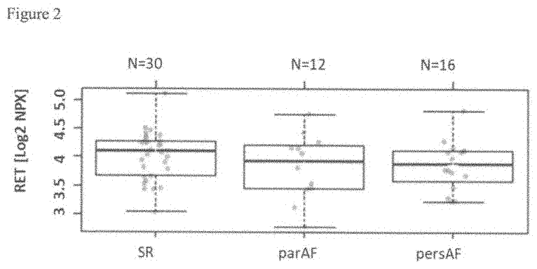

FIG. 2: Measurement of RET in Mapping study; Exploratory AFib panel; Patients with a history of atrial fibrillation undergoing open chest surgery and epicardial mapping of paroxysmal AF, persistent AF or SR (Mapping study). Circulating RET levels were assessed.

FIG. 3: Measurement of RET in SWISS AF study with Fazekas Score <2 (no) vs Fazekas Score≥2 (yes): Detection of WMLs/Prediction of the risk of silent stroke: Circulating RET levels were assessed.

### EXAMPLES

### Example 1: Differential Expression of RET in Cardiac Tissue of AF Patients

Differential RET expression levels have been determined in tissue samples from the right atrial appendage of n=40 patients. The right atrial appendage is associated to the heart right atrium.

RNAseq Analyses

Atrial tissue was sampled during open chest surgery because of CABG or valve surgery. Evidence of AF or SR (controls) was generated during surgery with simultaneous Endo-Epicardial High Density Activation Mapping. Patients with atrial fibrillation and controls were matched with regard to gender, age and comorbidities.

Atrial tissue samples were prepared for
AF patients, n=11 patients
control patients in SR; n=28 patients

Differential expression of RET (alias CDHF12, CDHR16, HSCR1, MEN2A, MEN2B, MTCA, PTC, RET-ELE1, RETS1) was determined in RNAseq analyses applying the algorithms RSEM and DESEQ2.

As shown in FIG. 1, RET expression was found to be downregulated in the analyzed atrial tissues of the 11 patients with persistent AF versus the 28 control patients in sinus rhythm. The fold change in expression (FC) was −1.24.

The altered expression of RET was determined in the damaged end organ, the right atrial appendage, part of the atrial tissue of the heart right atrium. RET mRNA levels were compared for patients with an ongoing atrial fibrillation event in course of surgery with control samples in sinus rhythm. The status of atrial fibrillation was the result of high density mapping of the atrial tissue Reduced RET mRNA levels were detected in atrial tissue samples with conduction disturbances as characterized by electrical mapping. Conductance disturbances may be caused by fat infiltration or by interstitial fibrosis. The observed differential expression of RET in atrial tissue of patients suffering from atrial fibrillation supports, that RET is released in the circulation from the myocardium, in particular from the heart atrium, in setail the right atrial appendage and reduced serum/plasma titers assist the detection of episodes of AF.

It is concluded, that RET is released from the heart into the blood and may aid the detection of AF episodes and predict the risk of developing AF related stroke.

### Example 2: Assessment of AF with Circulating RET

The MAPPING study related to patients undergoing open chest surgery. EDTA plasma samples were obtained before anesthesia and surgery. Patients were electrophysiologically characterized using high-density epicardial mapping with multi-electrode arrays (high density mapping).

Circulating RET protein levels have been determined in 14 patients with paroxysmal atrial fibrillation, 16 patients with persistent atrial fibrillation and 30 controls, matched to best possible (on age, gender, comorbidities). RET was determined in samples of the MAPPING study.

Measurements were performed in 30 patients with sinus rhythm (SR), in 12 patients with paroxysmal arterial fibrillation (parAF) and in 16 persistent arterial fibrillation (persAF).

FIG. 2 shows that RET titers are reduced in patients with paroxysmal AF and in patients with persAF in comparison to patients in SR (AUC 0.59 and AUC 0.62 respectively). Therefore, RET could be used for aid in diagnosis of patients with different types of AF. Reduced RET values would indicate a higher probability of AF.

### Example 3: Prediction of Clinical Stroke with Circulating RET

RET in the assessment of clinical stroke provides a method to

1. Predicting the risk of stroke in patients with atrial fibrillation based on circulating RET levels in serum/plasma (BeatAF study, SWISSAF study, Table 1)

2. Improving the prediction of clinical accuracy of clinical stroke risk scores based on circulating RET levels in serum/plasma (e.g. CHA₂DS₂-VASc, CHADS₂, ABC score)

The ability of circulating RET to predict the risk for the occurrence of stroke was assessed in two prospective, multicentric registry studies of patients with documented atrial fibrillation with similar inclusion criteria, the Beat AF and the SWISS AF study (Conen D., Forum Med Suisse 2012; 12:860-862; Conen et al., Swiss Med Wkly. 2017; 147). Patients of the SWISS AF cohort have a median age of 74 years, a rate of prior clinical strokes or TIA of 20%, a rate of vascular diseases of 34% and a history of diabetes of 17%. Patients of the Beat AF cohort have a median age of 70 years, a rate of prior clinical strokes or TIA of 16%, a rate of vascular diseases of 24% and a history of diabetes of 14%.

RET was measured using a stratified case cohort design. For each of the patients, which experienced a stroke during a follow up period of 5 years (70 clinical stroke patients in the Beat AF) 3 years (66 clinical stroke patients in the SWISS AF) (“events”), 1 control per event was selected.

RET were measured using the Olink platform. Therefor no absolute concentration values are available and can be reported. Results are reported on an arbitrary signal scale (NPX).

In order to quantify the univariate prognostic value of RET proportional hazard models were used with the outcome stroke.

The univariate prognostic performance of RET was assessed by two different incorporations of the prognostic information given by RET.

The first proportional hazard model included RET binarized at the median and therefore comparing the risk of patients with RET below or equal to the median versus patient with RET above the median.

The second proportional hazard model included the original RET levels but transformed to a log 2 scale. The log 2 transformation was performed in order to enable a better model calibration.

Because the estimates from a naïve proportional hazard model on the case control cohort would be biased (due to the altered proportion of cases to controls), a weighted proportional hazard model was used. Weights are based on the inverse probability for each patient to be selected for the case control cohort. In order to get estimates for the absolute survival rates in the two groups based on the dichotomized baseline RET measurement (<=median vs >median) a weighted version of the Kaplan-Meier plot was created.

In order to assess the ability of RET to improve existing risk scores for the prognosis of stroke the CHADS₂ the CHA₂DS₂-VASc and the ABC score were extended by RET (log 2 transformed). Extension was done by creating a portioned hazard model including RET and the respective risk score as independent variables.

The c-indices of the CHADS₂, the CHA₂DS₂-VASc and ABC score were compared to the c-indices of these extended models. For the calculation of the c-index in the case-cohort setting a weighted version of the c-index was used as proposed in Ganna (2011).

### Example 4. Prediction of Silent Stroke with Circulating RET

Data in the SWISS-AF data shows that RET correlates with existence of white matter lesions (WML) in patients. The extent of matter lesions can be expressed by the Fazekas score (Fazekas, J B Chawluk, A Alavi, H I Hurtig, and R A Zimmerman American Journal of Roentgenology 1987 149:2, 351-356). The Fazekas score is ranging from 0 to 3.0 indicates no WML, 1 mild WML, 2 moderate WML and 3 severe WML. In order to compare the association of RET with WML patients were classified in two groups, Fazekas Score <2 (no) vs Fazekas Score ≥2 (yes). FIG. 3 shows that RET is reduced in patients with moderate or severe WMLs versus patients with mild or no WMLs.

WML extent can be caused by clinical silent strokes (Wang Y, Liu G, Hong D, Chen F, Ji X, Cao G. White matter injury in ischemic stroke Prog Neurobiol. 2016; 141:45-60. doi:10.1016/j.pneurobio.2016.04005). This further advocates the usefulness of RET to predict the risk for clinical stroke.

The ability of circulating RET to discriminate between patients with Fazekas Score <2 (no) versus Fazekas Score ≥2 (yes) is indicated by the AUC of 0.64. White matter changes in the brain of dementia patients Advanced age and changes in WML scores have been described to be associated with severity of dementia in Alzheimers disease patients (Kao et al., 2019).

Age is also an important predictor of clinical stroke. Therefor it is plausible that data of significantly reduced RET levels in the circulation indicate not only moderate or severe WML, but also indicate age related brain diseases, e.g. vascular dementia.

Results

Table 1 shows the results of univariate weighted proportional hazard model including log transformed values of RET.

The association between the risk for experiencing a stroke with the baseline value of RET is highly significant.

The hazard ratio for RET implies a 0.39 fold higher risk for a stroke in the patients of the Beat AF study and a 0.43 fold higher risk for a stroke in patients of the SWISS AF cohort.

The results of the proportional hazard model including RET as log 2 transformed linear risk predictor suggest the log 2 transformed values RET are proportional to the risk for experiencing a stroke

TABLE 1
Measurement of circulating RET in Beat AF and in
SWISS AF patients; Case control of patients experiencing
a clinical stroke event in the follow up

| | |
|---|---|
| Beat AF | SWISS AF |

| | HR | AUC | p-Value | HR | AUC | p-Value |
|---|---|---|---|---|---|---|
| RET | 0.39 | 0.69 | 0.0007 | 0.43 | 0.67 | 0.0223 |

As demonstrated in Table 1, Beat AF study data show the surprising finding of highly significantly reduced levels of circulating RET titers in samples of patients with atrial fibrillation, that experienced a stroke in the follow up period. This finding was replicated in an independent study cohort (SWISS AF). Patients of the SWISS AF cohort have a higher number of risk factors for stroke including e.g. higher age, more comorbidities, shorter time until the experience of stroke versus patients of the Beat AF cohort.

Obviously circulating RET levels associate with the severity of the risk. The finding of significantly reduced RET levels in patients at risk of experiencing a stroke in the next years was consistent between the two cohorts (Beat AF and SWISS AF). It is remarkable that circulating RET levels indicate the risk of stroke independent of differences in the burden of comorbidities, which is different in the investigated cohorts.

As demonstrated in Table 1 reduced RET levels in the circulation associate with the severity of the risk of experiencing a clinical stroke.

These data suggest that RET can be used to assess the risk of stroke, to classify the disease, to assess the disease severity, to guide therapy (with objectives to therapy intensification/reduction), to predict disease outcome (risk prediction, e.g. stroke), therapy monitoring (e.g., effect of anticoagulation drugs on RET levels), therapy stratification (selection of therapy options).

Table 2 shows the estimated c-indexes of RET alone, of the CHADS₂, the CHA₂DS₂-VASc, the ABC score and of the weighted proportional hazard model combining the CHADS₂, the CHA₂DS₂VASc, the ABC score with RET (log 2) on the case cohort selection.

1. A method for predicting the risk of stroke and/or dementia in a subject, said method comprising
a) determining the amount of the biomarker RET (Rearranged during transfection) in a sample from the subject,
b) comparing the amount determined in step a) to a reference, and
c) predicting the risk of stroke and/or dementia.

2. The method of claim 1, wherein the subject suffers from atrial fibrillation.

3. The method of claim 1, wherein the risk of stroke is predicted and wherein the stroke is ischemic stroke.

4. The method of claim 1, wherein the risk of dementia is predicted, and wherein dementia is vascular dementia, Alzheimer's disease, dementia with Lewy bodies and/or frontotemporal dementia.

5. The method of claim 1, wherein an amount of RET lower than the reference is indicative for a subject who is at risk of stroke and/or dementia, and/or wherein an amount of RET larger than the reference is indicative for a subject who is not at risk of stroke and/or dementia.

6. The method of claim 1, wherein the risk of the subject to suffer from stroke and/or dementia in a subject within 1 to 10 years is predicted.

7. A computer-implemented method for predicting stroke and/or dementia in a subject, said method comprising
a) receiving at a processing unit a value for the amount of the biomarker RET (Rearranged during transfection) in a sample from the subject,
b) processing the value received in step (a) with the processing unit, wherein said processing comprises retrieving from a memory one or more threshold values for the amount of the biomarker RET and comparing the value received in step (a) with the one or more threshold values, and
c) providing a prediction of stroke and/or dementia via an output device, wherein said prediction is based on the results of step (b).

8. A method for improving the prediction accuracy of a clinical stroke risk score for a subject, comprising the steps of
a) determining the amount of the biomarker RET (Rearranged during transfection) in a sample from the subject, and
b) combining a value for the amount of the biomarker RET with the clinical stroke risk score, whereby the prediction accuracy of said clinical stroke risk score is improved.

9. A method for assessing the extent of white matter lesions in a subject, said method comprising
a) determining the amount of the biomarker RET (Rearranged during transfection) in a sample from the subject, and
b) assessing the extent of white matter lesions in a subject based on the amount determined in step a).

10. A method for monitoring the extent of white matter lesions and/or the cognitive function in a subject, comprising
a) determining the amount of the biomarker RET (Rearranged during transfection) in a first sample from the subject,
b) determining the amount of the biomarker RET (Rearranged during transfection) in a second sample from the subject which has been obtained after the first sample,
c) comparing the amount of the biomarker RET in the first sample to the amount of the biomarker RET in the second sample, and
d) monitoring the extent of white matter lesions and/or the cognitive function of the subject based on the results of step c).

11. A method for assessing whether a subject has experienced one or more silent strokes, said method comprising
a) determining the amount of the biomarker RET (Rearranged during transfection) in a sample from the subject,
b) comparing the amount determined in step a) to a reference, and
c) assessing whether a subject has experienced one or more silent strokes.

12. A method for diagnosing atrial fibrillation in a subject, said method comprising
a) determining the amount of the biomarker RET (Rearranged during transfection) in a sample from the subject,
b) comparing the amount determined in step a) to a reference, and
c) diagnosing atrial fibrillation.

13. The method of claim 1, wherein the sample is a blood, serum or plasma sample, or wherein the sample is a heart or neural tissue sample.

14. (canceled)

15. The method of claim 1, wherein
i. the biomarker RET is the RET polypeptide,
ii. the subject is human,
iii. the subject is 65 years or older, and/or
iv. the subject has no known history of stroke and/or TIA (transient ischemic attack).
